(19) United States
(12) Patent Application Publication
(10) Pub. No.: US 2019/0000913 A1
(43) Pub. Date: Jan. 3, 2019

(54) NUTRITIONAL COMPOSITIONS COMPRISING HYDROLYZED PROTEIN AND USES THEREOF

(71) Applicant: ABBOTT LABORATORIES, Abbott Park, IL (US)

(72) Inventors: TAPAS DAS, Worthington, OH (US); MUSTAFA VURMA, Dublin, OH (US); CHRON-SI LAI, Blacklick, OH (US); PAUL JOHNS, Columbus, OH (US)

(73) Assignee: ABBOTT LABORATORIES, Abbott Park, IL (US)

(21) Appl. No.: 16/066,557

(22) PCT Filed: Dec. 27, 2016
(86) PCT No.: PCT/US2016/068669

§ 371 Date: Jun. 27, 2018

Related U.S. Application Data
(60) Provisional application No. 62/271,510, filed on Dec. 28, 2015.

(51) Int. Cl. A61K 38/01; A61P 1/00; A23L 33/18; A23L 33/00; A23L 33/125

(52) U.S. Cl. CPC A61K 38/018; A61P 1/00; A23V 2002/00; A23L 33/40; A23L 33/125; A23L 33/18

(57) ABSTRACT

Disclosed embodiments provide a method of enhancing maturation of a lung, brain, or both, in an infant. The method includes the step of administering to the infant a nutritional composition comprising a protein with a degree of hydrolysis of 10% to 75%. Also disclosed are methods for reducing the incidence of necrotizing enterocolitis in an individual. In addition, the disclosed embodiments provide a method for the prevention, delay of progression, or the treatment of a circulatory disorder characterized by inadequate blood flow to the brain or lung.

### CROSS REFERENCE TO RELATED APPLICATIONS

This application claims priority to and the benefit of U.S. Application No. 62/271,510, filed on Dec. 28, 2015, the entire content of which is incorporated herein by reference.

### FIELD

The present disclosure relates to a method of enhancing maturation of a lung, brain, or both in an infant as well as a method for reducing the risk of necrotizing enterocolitis (NEC). In particular, the method includes the step of administering a nutritional composition to the infant.

### BACKGROUND

Infants born preterm have a variety of specific medical needs such as complications arising from immaturity of a variety of organs including the lung and brain. For example, infants with immature lungs often develop respiratory distress syndrome (“infant respiratory distress syndrome” or “IRDS”) caused by developmental insufficiency of surfactant production and structural immaturity in the lungs. The disorder can also result from a genetic problem with the production of surfactant-associated proteins. As such, IRDS affects about 1% of newborn term infants and is the leading cause of death in preterm infants. Currently, surfactant treatments, steroid treatments, and ventilation strategies are employed to improve development of the lungs in preterm infants. However, preterm infants born extremely early have lungs with small lung gas volumes and delicate lung tissue that is susceptible to injury when using ventilation methods. Thus, standard treatments pose a risk of postnatal injury. (Jobe, et al., “Lung Development and Function in Preterm Infants in the Surfactant Treatment Era,” Annual Review of Physiology, Vol. 62: 825-846 (2000)).

### SUMMARY

Disclosed herein are methods of enhancing maturation of a lung, brain, or both, as well as methods for reducing the incidence or risk of NEC, in an infant. Applicants have found that administration of particular hydrolyzed proteins provides unexpected nutritional and health benefits to infants, especially preterm infants and those in need of catch-up growth or development. In particular, blood flow to certain organs (e.g., lung, brain, or both) can be enhanced, as well as treating, reducing the incidence of, or delaying the progression of necrotizing enterocolitis, allowing for better nutrient availability and uptake. Increased blood flow, especially for preterm infants or those in need of catch-up growth, leads to improved maturation of organs, e.g., the lung and brain.

In a first exemplary embodiment, a method of enhancing maturation of a lung, brain, or both in an infant includes the step of administering a nutritional composition including a protein with a degree of hydrolysis of 10% to 75%, to an infant.

In a second exemplary embodiment, a method of reducing the risk of developing necrotizing enterocolitis is provided. The method comprises administering to an infant in need thereof a nutritional composition comprising a prophylactically effective amount of a protein having a degree of hydrolysis of 10% to 75%.

In a third exemplary embodiment, a method of treating or delaying the progression of necrotizing enterocolitis is provided. The method comprises administering to an infant in need thereof a nutritional composition comprising a protein having a degree of hydrolysis of 10% to 75%.

In a fourth exemplary embodiment, a method for the prevention, delay of progression, or the treatment of a circulatory disorder characterized by inadequate blood flow to the brain, lung, or both is provided. The method comprises administering to an individual in need thereof a nutritional composition comprising a therapeutically effective amount of a dipeptidyl-peptidase-4 (DPP-IV) inhibiting protein. The protein has a degree of hydrolysis of 10% to 75%.

In a fifth exemplary embodiment, a method of treating tissue inflammation in at least one of the gut, lung, and brain of an individual in need thereof is provided. The method comprises administering to the individual a nutritional composition comprising a protein with a degree of hydrolysis of 10% to 75%. After administration the tissue inflammation in at least one of the gut, lung, and brain of the individual is reduced.

### BRIEF DESCRIPTION OF THE DRAWINGS

FIG. 1 shows the results of DPP-IV inhibition as a function of molecular weight for several exemplary proteins.

FIG. 2 shows the results of DPP-IV inhibition as a function of average peptide chain length for several exemplary proteins.

FIG. 3 shows the results of DPP-IV inhibition as a function of average peptide chain length for several exemplary proteins.

FIG. 4 shows the effects of administration of a nutritional composition comprising a hydrolyzed protein on levels of surfactant A protein expression in hypoxia treated newborn mice.

FIG. 5 shows the results of IL-6 mRNA expression in the lung of hypoxia treated newborn mice (relative to RPLO).

FIG. 6 shows the results of IL-1b mRNA expression in the lung of hypoxia treated newborn mice (relative to RPLO).

FIG. 7 shows the results of IL-1b mRNA expression in the ileum of hypoxia treated newborn mice (relative to RPLO).

FIG. 8 shows the results of IL-6-12 mRNA expression in the ileum of hypoxia treated newborn mice (relative to RPLO).

FIG. 9 shows brain inflammation data on hypoxia treated mice that were dam reared (BM) or that were administered a NEC-F formula.

FIG. 10 shows the results of brain inflammation data on hypoxia treated mice obtained after administration of a modified human milk fortifier (HMF-M) and a modified human milk fortifier also including a predigested fat system (HMF-M-PDF).

### DETAILED DESCRIPTION

The general inventive concepts will be further described hereinafter in detail with reference to the accompanying drawings and various exemplary embodiments. One of ordinary skill in the art will appreciate that these exemplary embodiments only constitute a fraction of the possible embodiments encompassed by the general inventive concepts. As such, the scope of the present disclosure is by no means limited to the exemplary embodiments set forth herein.

The general inventive concepts are directed to nutritional compositions including hydrolyzed proteins. Applicants have found that, through the inclusion of a protein with a degree of hydrolysis of 10% to 75%, blood flow can be improved. In addition, glucagon-like peptide 2 (GLP-2) levels are increased. This combination leads to better blood flow and enhanced maturation (e.g., catch-up growth) for an infant, especially a preterm infant.

The enhanced blood flow can benefit an infant in a number of ways. In particular, administration of the hydrolyzed proteins, according to exemplary embodiments described herein, can reduce a level of dipeptidyl peptidase-4 (DPP-IV), thereby, lung maturation can be improved as indicated by an increase in lung surfactant A synthesis and/or a decrease in lung inflammatory cytokines. Without intending to be limited by theory, it is believed that the particular hydrolyzed proteins provided herein up-regulate the expression of genes that promote blood flow and lung maturation. The increased blood flow stimulates brain development via increasing supply of nutrients and helps to prevent, or reduce the risk of, necrotizing enterocolitis (NEC). Specifically, the nutritional compositions according to the general inventive concepts provide increased oxygen flow to the gastrointestinal (GI) tract. Thereby, the maturing gastrointestinal tract of the infant is exposed to a reduced level of hypoxia. This in turn results in reduced lipid oxidation and reduced inflammation (e.g., reduced levels of inflammatory cytokines) in the GI tract, including reduced reactive oxygen species (and reduced xanthine oxidase and TLR-4 expression), resulting in reduced incidence or risk of NEC.

The nutritional compositions disclosed herein provide the required nutritional benefits for growth and maturation to the infant, while providing the infant with the additional significant advantages of improved blood flow, improved lung development, improved brain development, reduced risk of necrotizing enterocolitis, and allowing more aggressive enteral nutritional feeding to allow a preterm infant to catch up on growth. The nutritional compositions as described herein may provide an individual, such as an infant, with dependable, high quality nutrition, as well as program the infant early in life such that the infant has a head start to a healthy body shape, improved lung development, improved brain development, and improved general overall health later in life. The nutritional compositions as described herein may provide the infant with nutritional benefits early in life that transcend into significant health benefits later in life, allowing the infant potentially to lead a longer, healthier life as a teenager and adult.

These and other features of the nutritional compositions and methods of the present disclosure, as well as some of the many other optional variations and additions, are described in detail hereafter.

The term “nutritional composition,” unless otherwise specified, refers to nutritional liquids, nutritional powders, nutritional solids, nutritional semi-liquids, semi-solids, nutritional supplements, nutritional tablets, and any other nutritional food product as known in the art. The nutritional powders may be reconstituted to form a nutritional liquid, all of which comprise one or more of fat, protein and carbohydrate, and are suitable for oral consumption by a human. Nutritional formulas include infant formulas.

The term “nutritional liquid,” as used herein, unless otherwise specified, refers to nutritional compositions in ready-to-drink liquid form, concentrated form, and nutritional liquids made by reconstituting the nutritional powders described herein prior to use.

The term “nutritional powder,” as used herein, unless otherwise specified, refers to nutritional compositions in flowable or scoopable form that can be reconstituted with water or another aqueous liquid prior to consumption and includes both spray dried and drymixed/dryblended powders.

The term “nutritional semi-solid,” as used herein, unless otherwise specified, refers to nutritional compositions that are intermediate in properties, such as rigidity, between solids and liquids. Some semi-solids examples include puddings, gelatins, and doughs.

The term “nutritional semi-liquid,” as used herein, unless otherwise specified, refers to nutritional compositions that are intermediate in properties, such as flow properties, between liquids and solids. Some semi-liquids examples include thick shakes and liquid gels.

The terms “hydrolyzed protein” or “protein hydrolysate” as used herein, unless otherwise specified, refer to a source of protein which has been subjected to a specific treatment whose primary purpose is to hydrolyze proteins. In this regard, it is conventional in this industry to refer to a protein source which has been subjected to a treatment whose primary purpose is to hydrolyze intact (or native) proteins to form hydrolyzed proteins, e.g., “whey protein hydrolysate.” In contrast, when a protein source has not been subjected to such a treatment, it is conventional practice to refer to this composition as a source of intact protein, or, more commonly, to say nothing about the hydrolysis of its protein.

One way of referring to the extent of hydrolysis of a hydrolyzed protein is by noting its Degree of Hydrolysis (DH). A protein with a DH value of, for example, 30 refers to protein in which 30% of the total peptide bonds within the protein are hydrolyzed (or that 30% of the protein's peptide bonds have been cleaved; e.g., if the intact protein contains 100 peptide bonds, and if 30 of these bonds are cleaved by the hydrolysis process, then the DH of the protein after hydrolysis is 30). As used herein, the degree of hydrolysis generally refers to the amount of amino nitrogen/total nitrogen in the protein.

The term “infant” as used herein, refers to individuals up to 36 months of age, actual or corrected. In certain embodiments, the term “infant” refers to individuals up to 36 months of age, actual or corrected, including individuals up to 12 months of age. The term “preterm infant,” as used herein, refers to an infant born prior to 36 weeks of gestation. The term “term infant,” as used herein, refers to an infant born at or after 36 weeks of gestation. The term “newborn infant,” as used herein, unless otherwise specified, refers to infants less than about 3 months of age, including infants from zero to about 2 weeks of age. The terms “infant” and “newborn infant” include both term and preterm infants.

The term “infant formula,” as used herein, unless otherwise specified, refers to liquid and solid nutritional compositions suitable for consumption by an infant. Unless otherwise specified herein, the term “infant formula” is intended to encompass term formulas, preterm infant formulas, and human milk fortifiers.

The term “preterm infant formula,” as used herein, unless otherwise specified, refers to liquid and solid nutritional compositions suitable for consumption by a preterm infant.

The term “later in life,” as used herein, refers to the period of life past infancy.

The terms “susceptible to,” and “at risk of,” as used herein, are used interchangeably to refer to individuals having little resistance to a certain condition or disease, including being genetically predisposed, having a family history of, and/or having symptoms of the condition or disease. In certain exemplary embodiments, the terms refer to a premature infant or an individual in need of catch-up growth. The terms are intended to refer to an individual with a greater need or are at an increased risk as compared to the general population or subset thereof.

All percentages, parts and ratios as used herein, are by weight of the total composition, unless otherwise specified. All such weights, as they pertain to listed ingredients, are based on the active level and, therefore, do not include solvents or by-products that may be included in commercially available materials, unless otherwise specified.

Numerical ranges as used herein are intended to include every number and subset of numbers within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

### Product Form

The nutritional compositions of the present disclosure may be formulated and administered in any known or otherwise suitable oral product form. Any solid, liquid, semi-solid, semi-liquid or powder form, including combinations or variations thereof, are suitable for use herein, provided that such forms allow for safe and effective oral delivery to the individual of the essential ingredients as also defined herein.

Specific non-limiting examples of product forms suitable for use with products and methods disclosed herein include, for example, liquid and powder preterm infant formulas, liquid and powder infant formulas, liquid and powder elemental and semi-elemental formulas, and liquid and powder human milk fortifiers.

The nutritional compositions of the present disclosure are desirably formulated as dietary product forms, which are defined herein as those embodiments comprising the ingredients of the present disclosure in a product form that then contains at least one of fat, protein, and carbohydrate.

The nutritional compositions may be formulated with sufficient kinds and amounts of nutrients to provide a sole, primary, or supplemental source of nutrition, or to provide a specialized nutritional composition for use in individuals such as infants afflicted with specific diseases or conditions or with a targeted nutritional benefit.

Desirably, the nutritional compositions include infant formulas formulated for both term and preterm infants. Desirably, the infant formula is formulated for feeding to infants within the first few days, weeks, or months following birth, and including for feeding to infants from age zero to one year, including zero to six months, including zero to four months, and including zero to two months. In some embodiments, the infant formulas are for feeding to newborn infants in the first few weeks of life, including birth to four weeks of life, including birth to three weeks of life, including birth to two weeks of life, and including birth to the first week of life. It is to be understood that the administration of the infant formulas of the present disclosure is not limited to administration during only the first six months following birth, but may be administered to older infants as well.

### Nutritional Liquids

Nutritional liquids include both concentrated and ready-to-feed nutritional liquids. These nutritional liquids include liquids formulated as suspensions, emulsions or clear or substantially clear liquids.

Nutritional emulsions suitable for use include aqueous emulsions comprising proteins, fats, and carbohydrates. These emulsions are generally flowable or drinkable liquids at from about 1° C. to about 25° C. and may be in the form of oil-in-water, water-in-oil, or complex aqueous emulsions, although such emulsions are most typically in the form of oil-in-water emulsions having a continuous aqueous phase and a discontinuous oil phase.

The nutritional liquids include liquids which are shelf stable. The nutritional liquids include liquids which contain up to about 95% by weight of water, including from about 50% to about 95%, also including from about 60% to about 90%, and also including from about 70% to about 85%, of water by weight of the nutritional liquid.

Although the serving size for the nutritional liquid can vary depending upon a number of variables, a typical serving sizes include those which are at least about 2 mL, or even at least about 5 mL, or even at least about 10 mL, or even at least about 25 mL, including ranges from about 2 mL to about 300 mL, including from about 100 mL to about 300 mL, from about 4 mL to about 250 mL, from about 150 mL to about 250 mL, and from about 10 mL to about 240 mL.

The nutritional emulsions may have a caloric density tailored to the nutritional needs of the ultimate user, although in most instances the emulsions comprise generally at least 19 kcal/fl oz (660 kcal/liter), more typically from about 20 kcal/fl oz (675-680 kcal/liter) to about 25 kcal/fl oz (820 kcal/liter), even more typically from about 20 kcal/fl oz (675-680 kcal/liter) to about 24 kcal/fl oz (800-810 kcal/liter). Generally, the 22-24 kcal/fl oz (740-810 kcal/liter) formulas are more commonly used in preterm or low birth weight infants, and the 20-21 kcal/fl oz (675-680 to 700 kcal/liter) formulas are more often used in term infants. In some embodiments, the emulsion may have a caloric density of from about 100 kcal/liter to about 660 kcal/liter, including from about 150 kcal/liter to about 500 kcal/liter.

In certain embodiments, the nutritional composition is a human milk fortifier, including a concentrated human milk fortifier. Human milk or other infant formula, after fortification with a concentrated liquid human milk fortifier will most typically have a caloric density ranging from about 19 kcal/fl oz (0.64 kcal/ml) to about 26.7 kcal/fl oz (0.9 kcal/ml), with the 22-25 oz formulations (0.74-0.84 kcal/ml) being more useful in preterm infants, and the 19-21 kcal/fl oz (0.64-0.71 kcal/ml) formulations more useful for term infants.

Concentrated liquid human milk fortifiers are generally formulated to have a caloric density of at least about 1.25 kcal/nil (37 kcal/fl oz), including from about 1.4 kcal/ml (42 kcal/fl oz) to about 5 kcal/ml (149 kcal/fl oz), and also including from about 1.5 kcal/ml (44 kcal/fl oz) to about 2.5 kcal/ml (74 kcal/fl oz), and also including from about 1.9 kcal/ml. (56 kcal/fl oz) to about 2.0 kcal/ml (59 kcal/fl oz).

### Nutritional Powders

The nutritional powders include powders in the form of flowable or substantially flowable particulate compositions, or at least particulate compositions. Particularly suitable nutritional powder forms include spray dried, agglomerated or dryblended powder compositions, or combinations thereof, or powders prepared by other suitable methods. The compositions can easily be scooped and measured with a spoon or similar other device, wherein the compositions may easily be reconstituted with a suitable aqueous liquid, typically water, to form a nutritional liquid, such as an infant formula, for immediate oral or enteral use. In this context, “immediate” use generally means within about 48 hours, most typically within about 24 hours, preferably right after or within 20 minutes of reconstitution.

### Hydrolyzed Protein

The various embodiments of the nutritional compositions described herein preferably include a protein with a degree of hydrolysis (DH) of 10% to 75%. In certain exemplary embodiments, the nutritional compositions include a protein with a DH of 10% to 55%. In certain exemplary embodiments, the nutritional compositions include a protein with a DH of 15% to 70%. In certain exemplary embodiments, the nutritional compositions include a protein with a DH of 20% to 65%. In certain exemplary embodiments, the nutritional compositions include a protein with a DH of 25% to 60%. In certain exemplary embodiments, the nutritional compositions include a protein with a DH of 25% to 55%.

In certain exemplary embodiments, the nutritional compositions include at least about 0.1 grams of hydrolyzed protein per liter of nutritional composition, including at least about 1 g/liter to about 150 g/liter, including at least about 1 g/liter to about 80 g/liter and including at least about 5 g/liter about 50 g/liter. In certain exemplary embodiments, the hydrolyzed protein makes up substantially all of the protein component of the nutritional composition. In certain exemplary embodiments, the hydrolyzed protein is present in an amount of 1% to 75% by weight of the total protein, including amount of 3% to 50% by weight of the total protein. In certain exemplary embodiments, the hydrolyzed protein is present in an amount of 3% to 40% by weight of the total protein. In certain exemplary embodiments, the hydrolyzed protein is present in an amount of 3% to 30% by weight of the total protein. In certain exemplary embodiments, the hydrolyzed protein is present in an amount of 3% to 20% by weight of the total protein. In certain exemplary embodiments, the hydrolyzed protein is present in an amount of 3% to 10% by weight of the total protein. In certain exemplary embodiments, the hydrolyzed protein is present in an amount of 5% to 10% by weight of the total protein. In certain exemplary embodiments, the hydrolyzed protein is present in an amount of 5% to 50% by weight of the total protein. In certain exemplary embodiments, the hydrolyzed protein is present in an amount of 10% to 50% by weight of the total protein. In certain exemplary embodiments, the hydrolyzed protein is present in an amount of 20% to 50% by weight of the total protein. In certain exemplary embodiments, the hydrolyzed protein is present in an amount of 30% to 50% by weight of the total protein. In certain exemplary embodiments, the hydrolyzed protein is present in an amount of 40% to 50% by weight of the total protein. In certain exemplary embodiments, the hydrolyzed protein is present in an amount of 50% to 75% by weight of the total protein. In certain exemplary embodiments, the hydrolyzed protein is present in an amount of 50% to 90% by weight of the total protein. In certain exemplary embodiments, the hydrolyzed protein is present in an amount of 50% to 100% by weight of the total protein.

The hydrolyzed protein may be present in the nutritional composition in these or other amounts, so long as the amount used is effective in improving blood flow to an organ selected from the brain and the lung or in enhancing maturation of an infant's lung, brain, or both, or in treating, reducing the incidence of, or delaying the progression of necrotizing enterocolitis, or combinations thereof. The hydrolyzed proteins may be derived from any suitable source. In some embodiments, the hydrolyzed proteins are derived from casein, whey, soy, pea, rice, corn, wheat, canola, potato, among others.

While not wishing to be bound by theory, it is believed that administration of hydrolyzed proteins in accordance with the general inventive concepts will inhibit DPP-IV, either directly or otherwise, thereby leading to an increase in glucagon-like peptide 2 (GLP-2), which results in enhanced or improved circulation, thereby resulting in better blood flow and nutrient and oxygen delivery to the targeted areas (e.g., the brain, lung, and gut) allowing the targeted areas to mature more readily. Thus, improved or enhanced blood flow improves maturation of the subject organ (e.g., brain, lung).

The amount of protein with DH of 10% to 75% that is effective for use according to the disclosed methods may vary based on the age or nutritional needs of the particular individual. In certain exemplary embodiments, the determination of the amount of protein with DH of 10% to 75% that is effective in, for example, improving blood flow to an organ selected from the brain and the lung, or in enhancing maturation of an infant's lung, brain, or both, or in treating, reducing the incidence of, or delaying the progression of necrotizing enterocolitis, or combinations thereof, may be determined in an animal model (such as that described in more detail below) or in an in vitro assay.

The hydrolyzed proteins according to the disclosed embodiments may be included as a portion of the protein in various infant nutritional compositions including preterm formulas, term formulas, human milk fortifiers, high protein formulas, concentrated liquids, and reconstitutable powders. Non-limiting examples of products that are suitable for inclusion of the hydrolyzed protein according to the general inventive concepts include the Similac and Alimentum lines of infant products sold by Abbott Nutrition.

When the nutritional compositions of the present disclosure are in powder form, then the powder is intended for reconstitution to liquid prior to use to obtain the above-noted requirements. Likewise, when the infant formulas of the present disclosure are in a concentrated liquid form, then the concentrate is intended for dilution prior to use to obtain the requisite requirements. The infant formulas can also be formulated as ready-to-feed liquids already having the requisite requirements.

The nutritional compositions of the present disclosure are desirably administered to infants, including preterm, term, and newborn infants, in accordance with the methods described herein. Such methods may include feedings with the infant formulas in accordance with the daily formula intake volumes described herein.

The nutritional compositions may include any protein, carbohydrate, fat, or source thereof that is known for or otherwise suitable for use in an oral nutritional composition, provided that the macronutrient is safe and effective for oral administration to infants and is otherwise compatible with the other ingredients in the infant formula. The protein, carbohydrate, and fat can be adjusted as necessary by one skilled in the art based on the disclosure herein to obtain the desired caloric density and protein level.

Although total concentrations or amounts of the protein, carbohydrate, and fat may vary depending upon the product form (e.g., powder or ready-to-feed liquid) and targeted dietary needs of the intended user, such concentrations or amounts most typically fall within one of the embodied ranges described in the following table (each numerical value is preceded by the term “about”), inclusive of any other essential fat, protein, and or carbohydrate ingredients as described herein.

TABLE 1

| Nutrient % Total Cal. | Embodiment A | Embodiment B | Embodiment C |
|---|---|---|---|
| Carbohydrate | 0-98 | 2-96 | 10-75 |
| Protein | 0-98 | 2-96 | 5-70 |
| Fat | 0-98 | 2-96 | 20-85 |
| | Embodiment D | Embodiment E | Embodiment F |
| Carbohydrate | 30-50 | 25-50 | 25-50 |
| Protein | 15-35 | 10-30 | 5-30 |
| Fat | 35-55 | 1-20 | 2-20 |

The nutritional compositions may contain any percentage or amount of protein, carbohydrate, and fat described herein in combination with any disclosed percentage or amount of hydrolyzed proteins so long as the combination is safe and effective for oral administration to infants. In a particular embodiment, the nutritional composition (as administered) includes an amount of protein with DH of 10% to 75% that is effective in, for example, improving blood flow to an organ selected from the brain and the lung, or in enhancing maturation of an infant's lung or brain, or both, or in treating, reducing the incidence of, or delaying the progression of necrotizing enterocolitis, or combinations thereof.

### Protein

In addition to the hydrolyzed protein discussed above, any known or otherwise suitable protein or protein source may be included in the infant formulas of the present disclosure, provided that such proteins are suitable for feeding to infants, and in particular, newborn infants.

Non-limiting examples of suitable protein or sources thereof for use in the infant formulas include hydrolyzed, partially hydrolyzed or non-hydrolyzed proteins or protein sources, which may be derived from any known or otherwise suitable source such as milk (e.g., casein, whey), animal (e.g., meat, fish), cereal (e.g., rice, corn), vegetable (e.g., pea, soy), or combinations thereof. Non-limiting examples of such proteins include milk protein isolates, milk protein concentrates as described herein, casein protein isolates, extensively hydrolyzed casein, whey protein, sodium or calcium caseinates, whole cow milk, partially or completely defatted milk, soy protein isolates, soy protein concentrates, and so forth. The proteins for use herein can also include free amino acids known for use in nutritional compositions, non-limiting examples of which include L-alanine, L-aspartic acid, L-glutamic acid, glycine, L-histidine, L-isoleucine, L-leucine, L-phenylalanine, L-proline, L-serine, L-threonine, L-valine, L-tryptophan, L-glutamine, L-tyrosine, L-methionine, L-cysteine, taurine, L-arginine, L-carnitine, and combinations thereof.

Suitable sources of fat for use in the infant formulas disclosed herein include any fat or fat source that is suitable for use in an oral nutritional composition and is compatible with the essential elements and features of such products, provided that such fats are suitable for feeding to infants.

Non-limiting examples of suitable fats or sources thereof for use in the infant formulas described herein include coconut oil, fractionated coconut oil, soybean oil, corn oil, olive oil, safflower oil, high oleic safflower oil, high GLA-safflower oil, oleic acids, MCT oil (medium chain triglycerides), sunflower oil, high oleic sunflower oil, structured triglycerides, palm and palm kernel oils, palm olein, canola oil, flaxseed oil, borage oil, evening primrose oil, blackcurrant seed oil, transgenic oil sources, marine oils (e.g., tuna, sardine), fish oils, fungal oils, algae oils, cottonseed oils, and combinations thereof. In one embodiment, suitable fats or sources thereof include oils and oil blends including long chain polyunsaturated fatty acids (LC-PUFAs). Some non-limiting specific polyunsaturated acids for inclusion include, for example, docosahexaenoic acid (DHA), arachidonic acid (ARA), eicosapentaenoic acid (EPA), linoleic acid (LA), and the like. Non-limiting sources of arachidonic acid and docosahexaenoic acid include marine oil, egg derived oils, fungal oil, algal oil, and combinations thereof. Particularly preferred fat sources include high oleic safflower oil, soy oil, and coconut oils, which may all be used in combination with ARA and/or DHA oil. In one preferred embodiment, the infant formula included a combination of high oleic safflower oil, soy oil, and coconut oil, in combination with ARA oil and DHA oil.

### Carbohydrate

Carbohydrates suitable for use in the nutritional compositions include any carbohydrates that are suitable for use in an oral nutritional composition, such as an infant formula, and that are compatible with the essential elements and features of such product.

Non-limiting examples of suitable carbohydrates or sources thereof for use in the infant formulas described herein include maltodextrin, hydrolyzed, intact, or modified starch or cornstarch, glucose polymers, corn syrup, corn syrup solids, rice-derived carbohydrates, rice syrup, pea-derived carbohydrates, potato-derived carbohydrates, tapioca, sucrose, glucose, fructose, lactose, high fructose corn syrup, honey, sugar alcohols (e.g., maltitol, erythritol, sorbitol), artificial sweeteners (e.g., sucralose, acesulfame potassium, stevia), indigestible oligosaccharides such as fructooligosaccharides (FOS), and combinations thereof. In one embodiment, the carbohydrate includes a maltodextrin having a DE value of less than 20. One preferred carbohydrate is lactose.

### Optional Ingredients

In certain exemplary embodiments, the nutritional compositions of the present disclosure include one or more nucleotides. The nucleotides may be used alone or in combination with any of the other nutritional components as described herein. Administration or consumption of nucleotides can reduce long term adverse health effects of diet in an individual, including long term obesity. “Nucleotides” as used herein includes nucleotides, nucleosides, nucleobases, and combinations thereof, unless otherwise specified in a particular embodiment. Suitable nucleotides include nucleotides with purine bases, pyrimidine bases, ribose and deoxyribose sugars. “Nucleotides” include nucleotides in monophosphate, diphosphate, or triphosphate form. “Nucleotides” also include nucleotides in monomeric, dimeric, or polymeric (including RNA and DNA) form. Also included in the term “nucleotides” are those nucleotides present in the infant formula as a free acid or in the form of a salt, preferably a monosodium salt.

Suitable specific nucleotides and nucleosides for use in the nutritional compositions include one or more of 3′-deoxyadenosine, cytidine 5′-monophosphate, disodium guanosine 5′ monophosphate, disodium uridine 5′ monophosphate, uridine 5′-monophosphate, adenosine 5′-monophosphate, and guanosine 5′-1-monophosphate, Of these, particularly preferred nucleotides include cytidine 5′ monophosphate, disodium guanosine 5′ monophosphate, disodium uridine 5′ monophosphate, adenosine 5′ monophosphate, and combinations thereof. In some embodiments, the nucleotides are in free form and include adenine, cytosine, uracil, guanine, and thymine.

In certain exemplary embodiments, the nutritional compositions of the present disclosure include a carotenoid alone or in combination with any of the other nutritional components as described herein. The nutritional compositions may include one or more carotenoids, and particularly, one or more of the carotenoids lutein, lycopene, zeaxanthin and beta-carotene. In particular embodiments, the nutritional compositions include the carotenoid lutein. Other carotenoids may also be included in the infant formulas as described herein. Carotenoids included in the infant formulas disclosed herein include those carotenoids which are from a natural source as well as those which are artificially synthesized.

### Other Optional Ingredients

The nutritional compositions of the present disclosure include nutritional compositions which include other optional ingredients that may modify the physical, chemical, aesthetic or processing characteristics of the products or serve as pharmaceutical or additional nutritional components when used in the targeted population. Many such optional ingredients are known or otherwise suitable for use in medical food or other nutritional compositions or pharmaceutical dosage forms and may also be used in the compositions herein, provided that such optional ingredients are safe for oral administration and are compatible with the essential and other ingredients in the selected product form.

Non-limiting examples of such optional ingredients include preservatives, anti-oxidants, emulsifying agents, buffers, fructooligosaccharides, galactooligosaccharides, human milk oligosaccharides and other prebiotics, pharmaceutical actives, additional nutrients as described herein, colorants, flavors, thickening agents and stabilizers, lubricants, and so forth, and combinations thereof.

A flowing agent or anti-caking agent may be included in the powder formulas as described herein to retard clumping or caking of the powder over time and to make a powder embodiment flow easily from its container. Any known flowing or anti-caking agents that are known or otherwise suitable for use in a nutritional powder or product form are suitable for use herein, non limiting examples of which include tricalcium phosphate, silicates, and combinations thereof. The concentration of the flowing agent or anti-caking agent in the nutritional composition varies depending upon the product form, the other selected ingredients, the desired flow properties, and so forth, but most typically range from about 0.1% to about 4%, including from about 0.5% to about 2%, by weight of the composition.

A stabilizer may also be included in the nutritional compositions. Any stabilizer that is known or otherwise suitable for use in a nutritional composition is also suitable for use herein, some non-limiting examples of which include gums such as gellan gum, carrageenan, and xanthan gum. The stabilizer may represent from about 0.1% to about 5.0%, including from about 0.5% to about 3%, including from about 0.7% to about 1.5%, by weight of the infant formula.

### Methods of Manufacture

The nutritional compositions of the present disclosure may be prepared by any known or otherwise effective manufacturing technique for preparing the selected product solid or liquid form. Many such techniques are known for any given product form such as nutritional liquids or powders and can easily be applied by one of ordinary skill in the art to the infant formulas described herein.

The nutritional compositions of the present disclosure can therefore be prepared by any of a variety of known or otherwise effective formulation or manufacturing methods. In one suitable manufacturing process, for example, at least two separate slurries are prepared, that are later blended together, heat treated, standardized, and either terminally sterilized to form a retort infant formula or aseptically processed and filled to form an aseptic infant formula. Alternately, the slurries can be blended together, heat treated, standardized, heat treated a second time, evaporated to remove water, and spray dried to form a powder infant formula.

The slurries formed may include a carbohydrate-mineral (CHO-MIN) slurry, a protein-water slurry (PIW), and a protein-in-fat (PIF) slurry. Initially, the CHO-MIN slurry is formed by dissolving selected carbohydrates (e.g., lactose, galactooligosaccharides, etc.) in heated water with agitation, followed by the addition of minerals (e.g., potassium citrate, magnesium chloride, potassium chloride, sodium chloride, choline chloride, etc.). Soy lecithin is then added to the CHO-MIN slurry. The resulting CHO-MIN slurry is held with continued heat and moderate agitation until it is later blended with the other prepared slurries. The PIF slurry is formed by heating and mixing the oil (e.g., high oleic safflower oil, soybean oil, coconut oil, monoglycerides, etc.) and emulsifier (e.g., soy lecithin), and then adding oil soluble vitamins, mixed carotenoids, protein (e.g., milk protein concentrate, milk protein hydrolysate, etc.), carrageenan (if any), calcium carbonate or tricalcium phosphate (if any), and ARA oil and DHA oil (in some embodiments) with continued heat and agitation. The resulting PIF slurry is held with continued heat and moderate agitation until it is later blended with the other prepared slurries. PIW is with the CHO-MIN slurry, and the PIF slurry is added under adequate agitation. The pH of the resulting blend is adjusted to 6.6-7.0, and the blend was held under moderate heated agitation. ARA oil and DHA oil is added at this stage in some embodiments. The ratio blends is assembled by blending target amounts of PIW, PIF and CHO/MIN, the blend is then heated and homogenized. Water soluble vitamins are added and the standardized ratio is either spray dried or diluted, filled in appropriate containers, then retorted.

The composition is then subjected to high-temperature short-time (HTST) processing, during which the composition is heat treated, emulsified and homogenized, and then cooled. Water soluble vitamins, any trace minerals and ascorbic acid are added, the pH is adjusted to the desired range if necessary, flavors (if any) are added, and water is added to achieve the desired total solid level. For aseptic infant formulas, the emulsion receives a second heat treatment through an aseptic processor, is cooled, and then aseptically packaged into suitable containers. For retort infant formulas, the emulsion is packaged into suitable containers and terminally sterilized. In some embodiments, the emulsions are heat-treated then spray dried to make a reconstitutable powder. This powder product can be agglomerated or dry blended with other heat labile nutrients.

The spray dried powder nutritional composition or dry-mixed powder nutritional composition may be prepared by any collection of known or otherwise effective techniques, suitable for making and formulating a nutritional powder. For example, when the powder infant formula is a spray-dried nutritional powder, the spray drying step may likewise include any spray drying technique that is known for or otherwise suitable for use in the production of nutritional powders. Many different spray drying methods and techniques are known for use in the nutrition field, all of which are suitable for use in the manufacture of the spray dried powder infant formulas herein. Following drying, the finished powder may be packaged into suitable containers.

### Methods of Use

The nutritional compositions of the present disclosure include infant formulas and human milk fortifiers that are orally administered to infants, including preterm or term infants. The infant formulas may be administered as a source of nutrition for infants, to prevent and/or reduce and/or minimize and/or eliminate the development and/or onset of necrotizing enterocolitis, and/or to enhance blood flow to an organ selected from the brain and lung, and/or to enhance maturation of the lungs, brain, or both. Generally, an increase or improvement in blood flow will improve maturation of an organ (e.g., brain, lung), especially in those in need of catch-up growth. One subclass of the general infant population that can effectively utilize the infant formulas described herein include those infants that are susceptible to, or at a risk of (at an elevated risk as compared to the general infant population) one or more of necrotizing enterocolitis and immaturity of an organ selected from the lung and brain. These infants who are susceptible to or at risk of having necrotizing enterocolitis and/or immaturity of an organ selected from the lung and brain are herein referred to as “in need of” assistance (or “in need thereof” as referring to the assistance needed) in combating necrotizing enterocolitis and/or combating organ development problems such as respiratory issues.

Based on the forgoing, because some of the method embodiments of the present disclosure are directed to specific subsets or subclasses of infants (that is, the subset or subclass of infants that are “in need” of assistance in addressing infant formula tolerance or respiratory issues) in those embodiments, not all infants can benefit from all method embodiments described herein as not all infants will fall within the subset or subclass of infants as described herein.

The infant formulas will typically be administered daily, at intake volumes suitable for the age of the infant. For instance, in certain exemplary embodiments, the methods of the present disclosure include methods of administering one or more of the formulas of the present disclosure to an infant at the average intake volumes described herein. In other embodiments, newborn infants are provided with increasing formula volumes during the initial weeks of life. Such volumes most typically range to up to about 100 mL/day on average during the first day or so of life; up to about 200 to about 700 mL/day, including from about 200 to about 600 mL/day, and also including from about 250 to about 500 mL/day, on average during the remainder of the three month newborn feeding period. It is to be understood, however, that such volumes can vary considerably depending upon the particular newborn infant and their unique nutritional needs during the initial weeks or months of life, as well as the specific nutrients and caloric density of the infant formula administered.

In certain exemplary embodiments, the methods of the present disclosure are directed to infants during the initial days, weeks or months of life. Desirably, in certain exemplary embodiments, the infant formulas described herein are administered to the infant for a duration of at least the first week of life, more desirably during at least the first two weeks of life, more desirably during at least the first one or two months of life, more desirably during at least the first four months of life, and more desirably during at least the first six months of life, and including up to the first year of life. Thereafter, the infant may be switched to a conventional infant formula, alone or in combination with human milk. It should be understood by one skilled in the art based on the disclosure herein that the infant formulas described herein can be used alone, or in combination with human breast milk, or in combination with other infant formulas.

The infant formulas used in the methods described herein, unless otherwise specified, are nutritional formulas and may be in any product form, including ready-to-feed liquids, concentrated liquids, reconstituted powders, and the like as described above. In embodiments where the infant formulas are in powder form, the method may further comprise reconstituting the powder with an aqueous vehicle, most typically water or human milk, to form the desired caloric density, which is then orally or enterally fed to the infant. The powdered formulas are reconstituted with a sufficient quantity of water or other suitable fluid such as human milk to produce the desired caloric density, as well as the desired feeding volume suitable for one infant feeding. The infant formulas may also be sterilized prior to use through retort or aseptic means.

In certain exemplary embodiments, the present disclosure is directed to a method of providing targeted nutrition to an infant. The method comprises administering to the infant one or more of the infant formulas of the present disclosure, including infant formulas having a protein with a degree of hydrolysis of 10% to 75%. Such methods include methods where the infant formulas are administered on a daily basis, including administration at the daily intake volumes as described hereinbefore. In certain exemplary embodiments, the infant to whom the formula is administered is a newborn infant, including a preterm infant.

In certain exemplary embodiments, the present disclosure is directed to a method of enhancing maturation of an infant's lungs, brain, or both (including therapeutic or prophylactic treatments). The method comprises administering to the infant one or more of the infant formulas of the present disclosure. In certain exemplary embodiments, the infant to whom the formula is administered is a newborn infant, including a preterm infant. In certain exemplary embodiments, the infant to whom the formula is administered has experienced, or is at risk of experiencing diminished or impeded maturation of an organ selected from the lungs, brain, or both.

In certain exemplary embodiments, the present disclosure is directed to a method of reducing the risk of developing necrotizing enterocolitis (including therapeutic or prophylactic treatments). The method comprises administering to the infant one or more of the infant formulas of the present disclosure, including infant formulas having a protein with a degree of hydrolysis of 10% to 75%. In certain exemplary embodiments, the infant to whom the formula is administered is a newborn infant, including a preterm infant. In certain exemplary embodiments, the infant to whom the formula is administered has experienced, or is at risk of experiencing necrotizing enterocolitis.

In certain exemplary embodiments, the present disclosure is directed to a method of treating or delaying the progression of necrotizing enterocolitis (including therapeutic or prophylactic treatments). The method comprises administering to the infant one or more of the infant formulas of the present disclosure, including infant formulas having a protein with a degree of hydrolysis of 10% to 75%. In certain exemplary embodiments, the infant to whom the formula is administered is a newborn infant, including a preterm infant. In certain exemplary embodiments, the infant to whom the formula is administered has experienced, or is at risk of experiencing necrotizing enterocolitis.

In certain exemplary embodiments, the present disclosure is directed to a method for the prevention, delay of progression, or the treatment of a circulatory disorder characterized by inadequate blood flow to the brain or lung. The method comprises administering to an individual in need thereof a nutritional composition comprising a therapeutically effective amount of a DPP-IV inhibiting protein wherein the protein has a degree of hydrolysis of 10% to 75%. In certain exemplary embodiments, the infant to whom the formula is administered is a newborn infant, including a preterm infant. In certain exemplary embodiments, the infant to whom the formula is administered has experienced, or is at risk of experiencing inadequate blood flow to the brain or lung.

In certain exemplary embodiments, administration of the nutritional compositions according to the general inventive concepts provides increased blood flow and nutrient distribution to the individual. In particular, administration of hydrolyzed proteins according to the general inventive concepts results in inhibition of DPP-IV. Thereby, a level of GLP-2 will increase, resulting in increased blood flow to certain organs, including the brain and lungs. In addition, inhibition of DPP-IV may also enhance oxygen flow to the intestines which results in a reduction in hypoxia, and corresponding reduction in lipid oxidation and inflammation, all of which are implicated in the onset and progression of necrotizing enterocolitis. Accordingly, administration of the hydrolyzed peptides according to the general inventive concepts provides a variety of health benefits, including enhanced maturation of organs including the lung and brain as well as reducing the incidence and severity of necrotizing enterocolitis.

In certain exemplary embodiments, the present disclosure is directed to improving lung maturation in an individual. In certain exemplary embodiments, individuals with improved lung maturation are identified by measuring the level of lung surfactant A protein synthesis from lung tissue in the individual both before and after administration of a nutritional composition, as disclosed herein, to the individual. Individuals with improved lung maturation are identified as those individuals exhibiting increased lung surfactant A protein synthesis following the administration. The level of lung surfactant A protein synthesis is measured by any method known in the art. In some embodiments, lung surfactant A protein synthesis is measured by western blot.

In certain exemplary embodiments, improved maturation of the lung, brain, or both, in an individual, and particularly, an infant, is identified by measuring changes in at least one biomarker related to maturation of the lung, brain, or both (e.g., lung surfactant A protein synthesis). The biomarker may be measured in a model organism following administration of a nutritional composition disclosed herein to the model organism. The model organism can be any known model organism for measuring these properties. In some embodiments, the model organism is a rodent, and, in particular, a mouse.

### Examples

The following examples illustrate specific embodiments and/or features of the infant formulas and methods of the present disclosure. The examples are given solely for the purpose of illustration and are not to be construed as limitations of the present disclosure, as many variations thereof are possible without departing from the spirit and scope of the disclosure. Unless otherwise specified, the retort sterilized formulas, which may be prepared in accordance with the manufacturing methods described herein, are ready-to-feed liquid formulas. All exemplified amounts are weight percentages based upon the total weight of the composition, unless otherwise specified. All ingredient amounts are listed as kilogram per 1000 kilogram batch of product, unless otherwise specified.

Whey protein concentrate was suspended 14.6 g/L in 0.025 M TRIS (pH 8.0). Pancreatin (Sigma P-7545) prepared at 6 g/L in 0.025M TRIS (pH 8.0), was added in a WPC volume to Pancreatin volume ratio of 9:1. The suspension was incubated at 37° C. for between 0 and 240 minutes. The protein hydrolysates were then tested for molecular weight (MW) and average peptide length. The results are listed in table 2 below.

TABLE 2

| | MW median of | Average | |
|---|---|---|---|
| Pancreatin | Protein | peptide chain | DPP-IV |
| digestion time, | Hydrolysate | length (amino | inhibition, % |
| minutes | (Daltons) | acids) | of control |
| 0 | 10,767 | 90 | 4 |
| 15 | 1813 | 15 | 68 |
| 30 | 1217 | 10 | 74 |
| 60 | 803 | 6.7 | 77 |
| 90 | 693 | 5.8 | 78 |
| 120 | 637 | 5.3 | 79 |
| 240 | 539 | 4.5 | 81 |

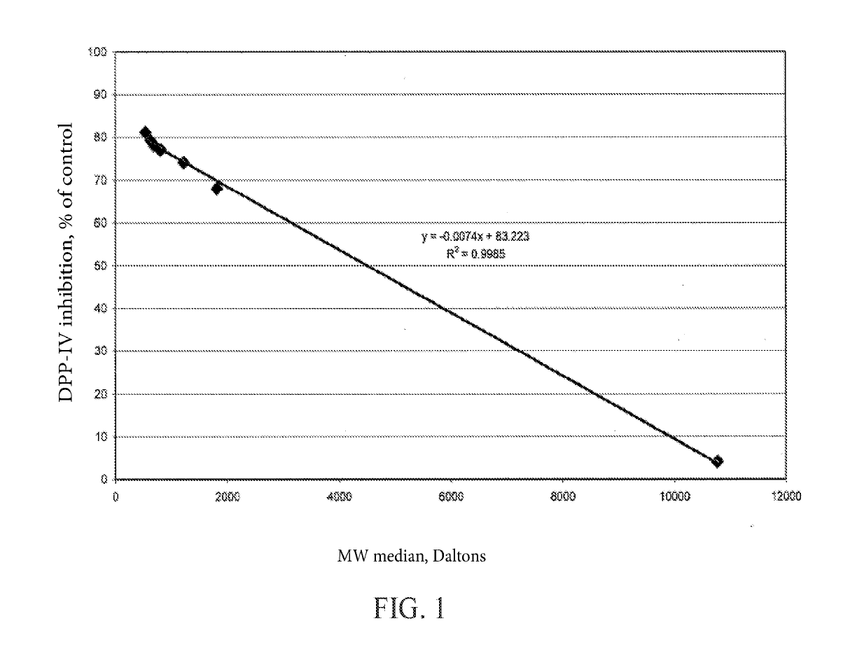

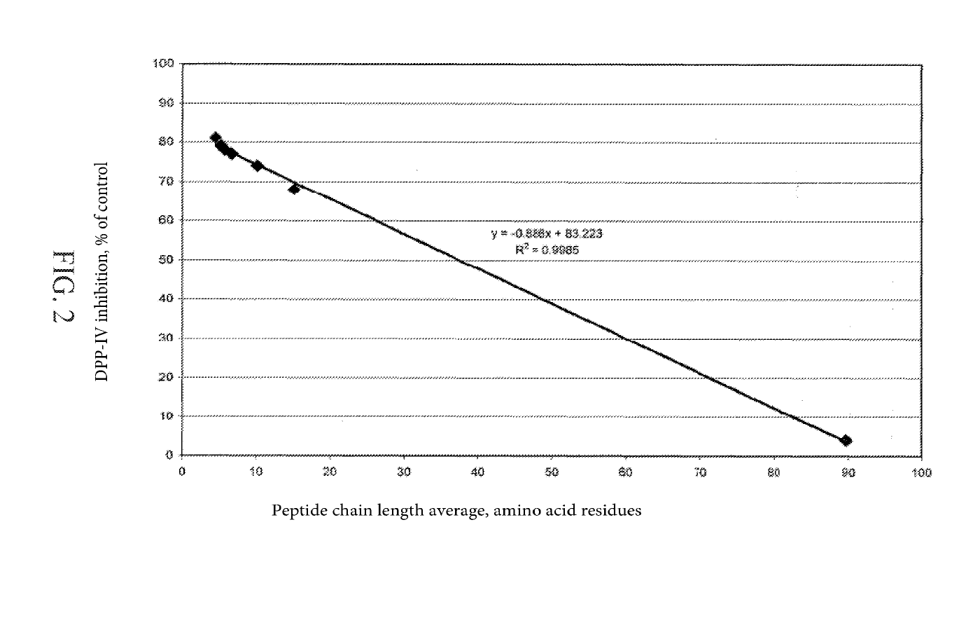

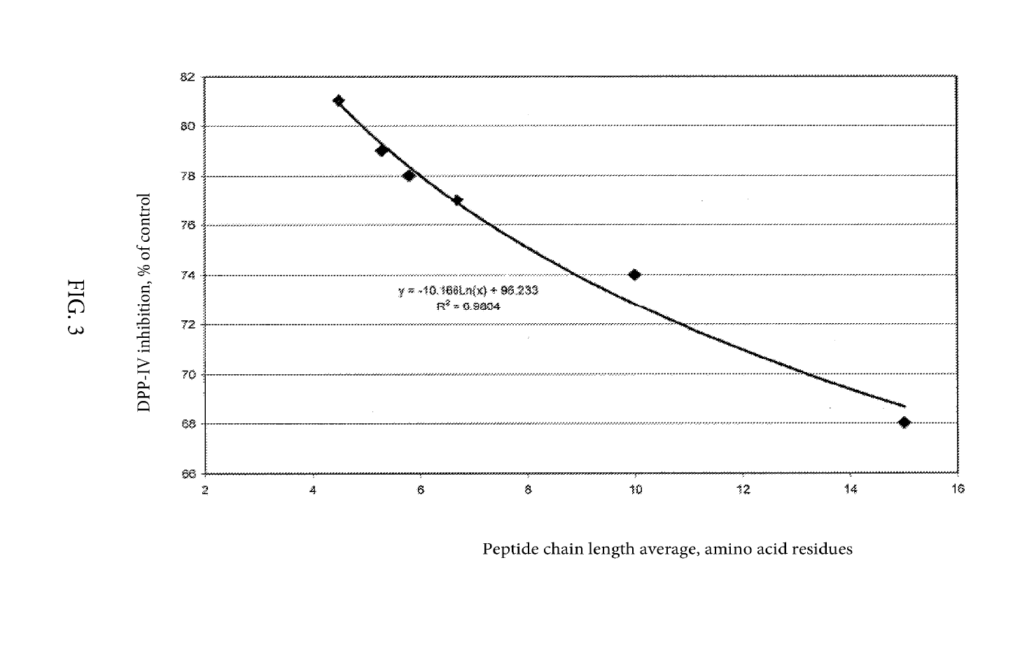

FIG. 1 shows the results of DPP-IV inhibition as a function of molecular weight (as measured by HPLC determination of the DPP-IV substrate Gly-Pro-p-nitroanilide and amount of p-nitroaniline released before and after hydrolysis of DPP-IV). FIG. 2 shows the results of DPP-IV inhibition as a function of average peptide chain length. FIG. 3 shows the results of DPP-IV inhibition as a function of average peptide chain length for a subset of the hydrolyzed proteins.

Table 3 is a bill of material for a Modified Human Milk Fortifier (HMF-M) including hydrolyzed protein in accordance with the general inventive concepts.

TABLE 3

| | Amount per | |
|---|---|---|
| Ingredients | 1000 lbs | UOM |
| Maltodextrin | 121.2 | lb |
| Casein Hydrolysate | 114.3 | lb |
| Soy Oil | 38.1 | lb |
| Calcium Phospahte | 15.8 | lb |
| Modified Corn Starch | 12.0 | lb |
| Potassium Citrate | 8.1 | lb |
| Ascorbic acid | 4.4 | lb |
| Magnesium Chloride | 3.5 | lb |
| Arachidonic Acid | 2.5 | lb |
| Docosahexaenoic Acid | 2.1 | lb |
| Potassium Chloride | 1.5 | lb |
| Water soluble vitamin | 600.0 | g |
| mineral premix | | |
| Sodium Chloride | 356.3 | g |
| Mixed carotenoids | 230.0 | g |
| Monoglycerides | 199.6 | g |
| Vitamin ADEK premix | 176.4 | g |
| Choline Chloride | 133.0 | g |
| Gellan Gum | 99.8 | g |
| Potassium hydroxide | as needed | |
| Ingredient Water | as needed | |

Necrotizing enterocolitis was induced in mice pups by supplementation of feed with enteric bacteria made from a stock created from a specimen obtained from an infant with surgical NEC. A mixture of a 2 to 1 blend of a model infant formula (Abbott Nutrition) and Esbilac (PetAg) canine milk replacer supplemented with enteric bacteria stock from an infant with surgical NEC (12.5 μl original stool slurry in 1 ml formula) via gavage five times/day. The seven to ten day old mice pups were divided into 4 groups. Group 1 (DR) was dam reared; Group 2 was fed a control formula including the NEC (NEC-F); Group 3 was fed a modified NEC formula (HMF-M) with hydrolyzed protein as described herein; Group 4 was fed a modified NEC formula including hydrolyzed protein and a modified fat system (HMF-M-PDF). The mice received hypoxia (5% O₂, 95% N₂) for 10 minutes in a hypoxic chamber (Billups-Rothenberg) twice daily for 4 days. Animals were fed 50 μl per gram of mouse body weight using a 24-French angiocatheter that was placed into the mouse esophagus under direct vision.

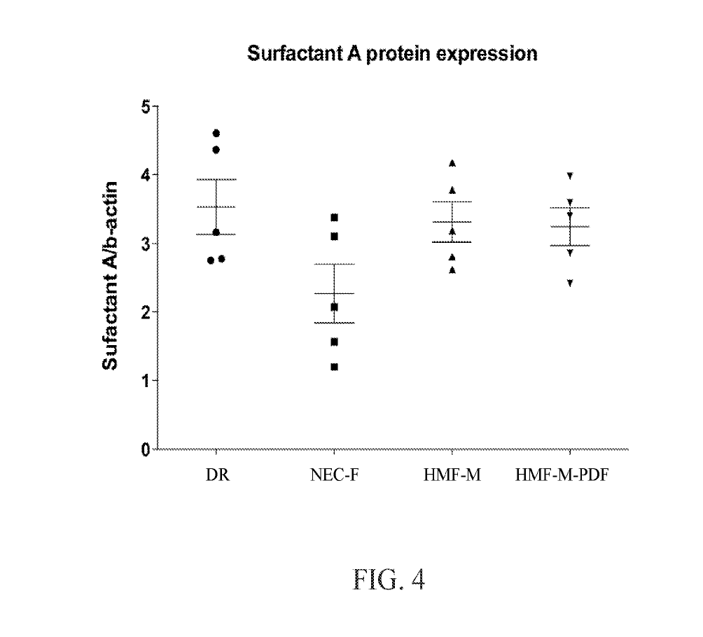

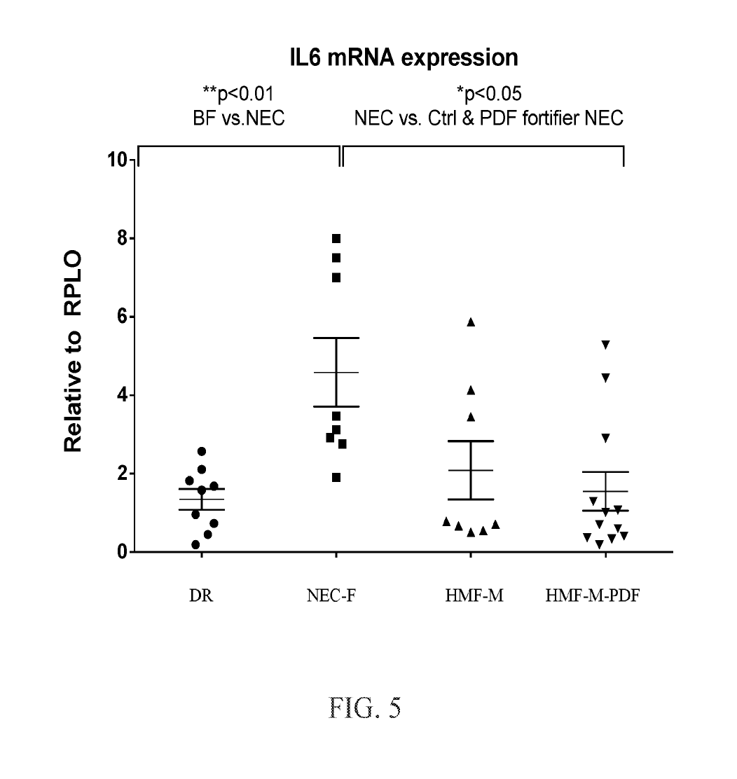

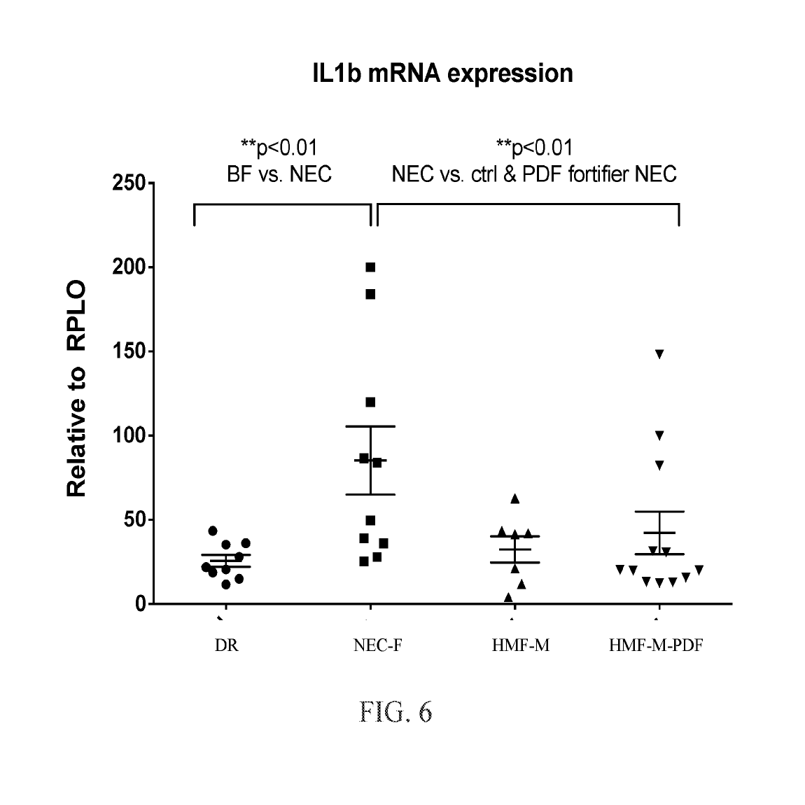

FIG. 4 shows the effects of the feeding on levels of surfactant A protein expression in the hypoxia treated newborn mice. Feeding hypoxia treated new born mice a formula containing hydrolyzed protein according to the disclosed embodiments reduces lung inflammation as evidenced by the measured levels of lung surfactant A protein synthesis. Lung surfactant protein A was quantified by Western blot (as relative to basal actin). FIG. 5 shows the results of IL-6 mRNA expression in the lung of hypoxia treated newborn mice (relative to RPLO). FIG. 6 shows the results of IL-1b mRNA expression in the lung of hypoxia treated newborn mice (relative to RPLO). Adding HMF-M or HMF+PDF to NEC formula reduced hypoxia treatment induced lung inflammation, and restored the lung surfactant protein A synthesis capacity of the mice.

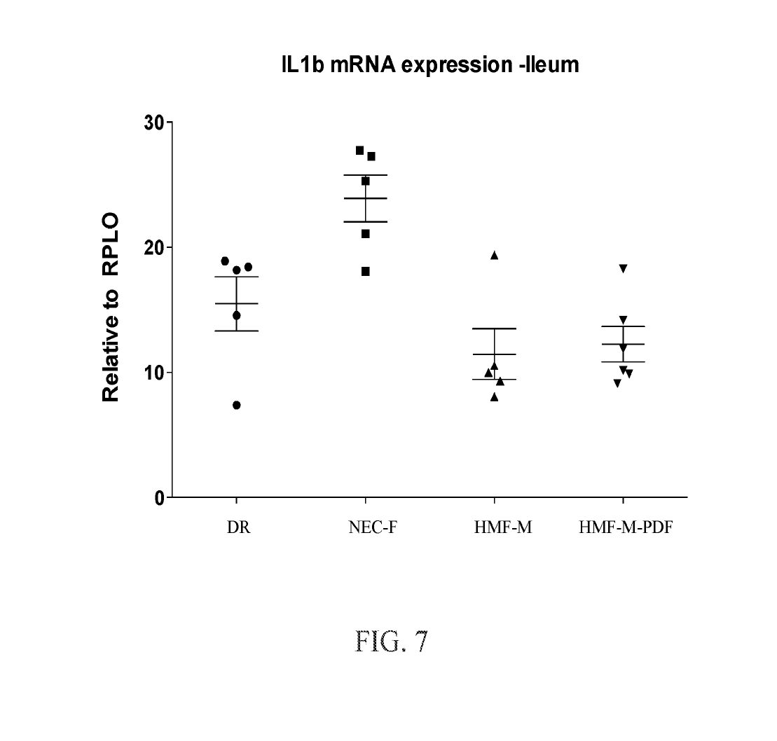

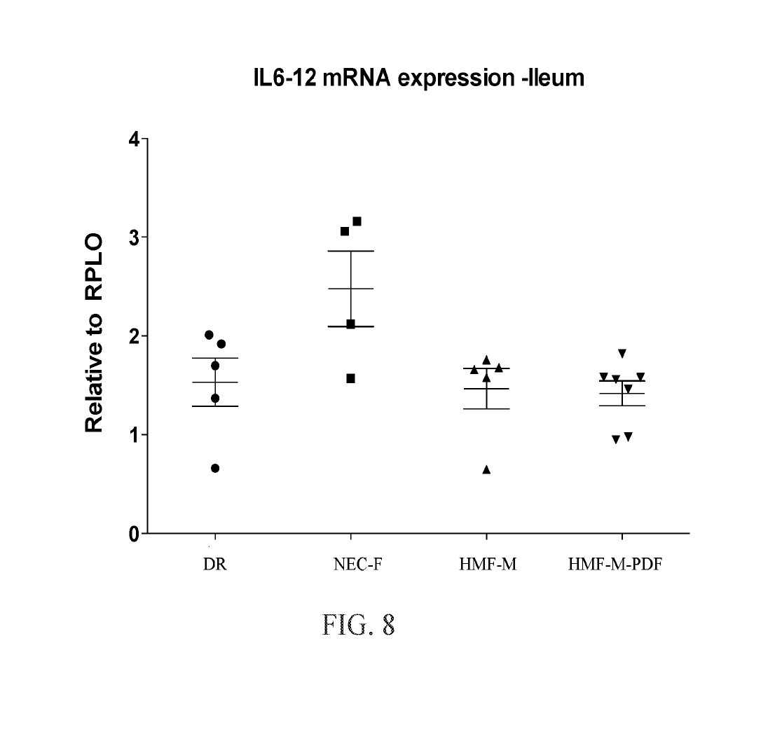

FIG. 7 shows the results of IL-1b mRNA expression in the ileum of hypoxia treated newborn mice (relative to RPLO). FIG. 8 shows the results of IL-6-12 mRNA expression in the ileum of hypoxia treated newborn mice (relative to RPLO). Adding HMF-M or HMF+PDF to NEC formula effectively lowered intestinal inflammation of the mice. Hydrolyzed proteins according to the disclosed embodiments appear to stimulate gut development by 1) providing easy to digest/absorb glutamine and glutamic acid and/or 2) improving intestinal blood flow by effectively increasing circulating GLP-2 in the treated mice.

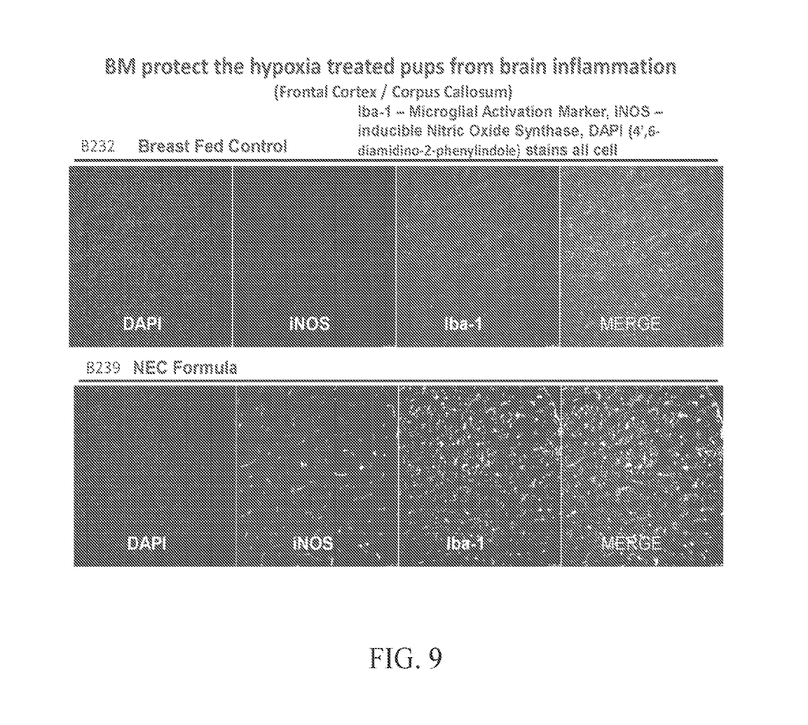

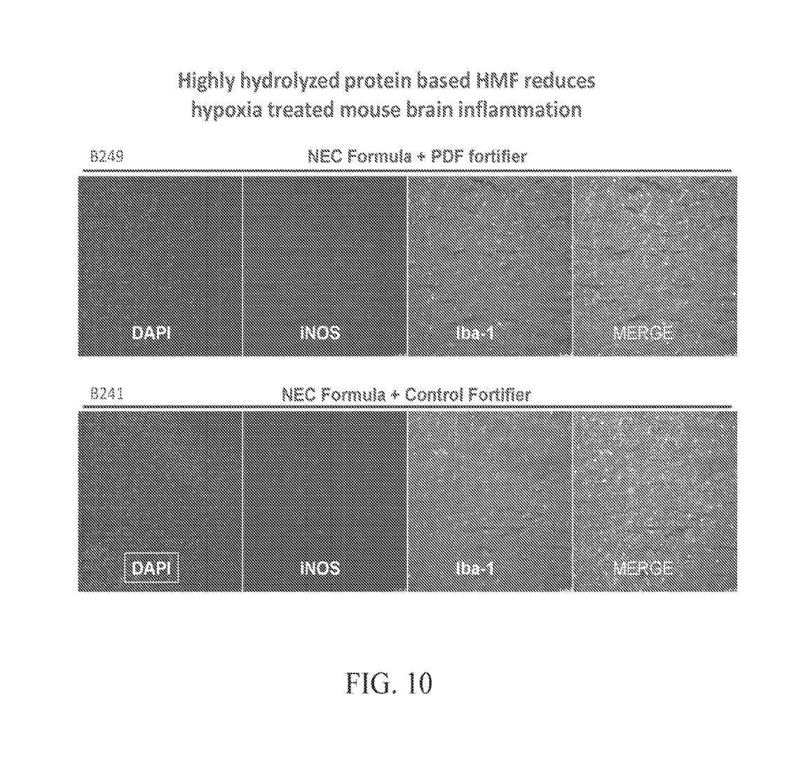

FIGS. 9 and 10 show brain inflammation data for hypoxia treated mice that were dan reared (BM), NEC formula (NEC-F), a modified human milk fortifier (HMF-M) and a modified human milk fortifier also including a predigested fat system (HMF-M-PDF). The expression of the pro-inflammatory cytokines were determined by quantitative real-time polymerase chain reaction (qRT-PCR) (Good M, Siggers R H, Sodhi C P, Afrazi A, Alkhudari F, Egan C E, Neal M D, Yazji I, Jia H, Lin J, Branca M F, Ma C, Prindle T, Grant Z, Shah S, Slagle D, Paredes J, Ozolek J, Gittes G K, Hackam D J. Amniotic fluid inhibits Toll-like receptor 4 signaling in the fetal and neonatal intestinal epithelium. Proceedings of the National Academy of Sciences. 2012; 109(28):11330-5. doi: 10.1073/pnas.1200856109.) Adding HMF-M and HMF+PDF to NEC formula reduced the number of iNOS and Iba-1 expressing cells in the hypoxia-treated pups brains, which suggests a lower level of oxidative stress and inflammation.

Although the present disclosure has been described with reference to specific embodiments, it should be understood that the limitations of the described embodiments are provided merely for purpose of illustration and are not intended to limit the present invention and associated general inventive concepts. Instead, the scope of the present invention is defined by the appended claims, and all variations and equivalents that fall within the range of the claims are intended to be embraced therein. Thus, other embodiments than the specific exemplary ones described herein are equally possible within the scope of these appended claims.

The various embodiments of the nutritional compositions of the present disclosure may also be substantially free of any optional or selected essential ingredient or feature described herein, provided that the remaining infant formulas still contain all of the required ingredients or features as described herein. In this context, and unless otherwise specified, the term “substantially free” means that the selected infant formulas contain less than a functional amount of the optional ingredient, typically less than 1%, including less than 0.5%, including less than 0.1%, and also including zero percent, by weight of such optional or selected essential ingredient.

The nutritional compositions and methods of the present disclosure may comprise, consist of, or consist essentially of the essential elements of the products and methods as described herein, as well as any additional or optional element described herein or otherwise useful in nutritional infant formula applications or other applications.

To the extent that the terms “includes,” “including,” “contains,” or “containing” are used in the specification or the claims, they are intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. Also, to the extent that the terms “in” or “into” are used in the specification or the claims, it is intended to additionally mean “on” or “onto.”

What is claimed is:

1. A method of improving blood flow to an organ selected from the brain and the lung in an individual in need thereof, the method comprising administering to the individual in need thereof a nutritional composition comprising a protein with a degree of hydrolysis of 10% to 75%, and wherein after administration the individual's blood flow to the selected organ is increased.

2. The method of claim 1, wherein the individual in need thereof is a preterm infant.

3. The method of any preceding claim, wherein the nutritional composition is a human milk fortifier.

4. The method of claim 3, wherein the nutritional composition is a powdered human milk fortifier.

5. The method of claim 1, wherein the nutritional composition is an infant formula.

6. The method of claim 5, wherein the nutritional composition is a preterm infant formula.

7. The method of claim 1, wherein the nutritional composition further comprises a carbohydrate.

8. A method of reducing the risk of developing necrotizing enterocolitis comprising administering to an infant in need thereof a nutritional composition comprising a prophylactically effective amount of a protein having a degree of hydrolysis of 10% to 75%.

9. A method of treating or delaying the progression of necrotizing enterocolitis comprising administering to an infant in need thereof a nutritional composition comprising a protein having a degree of hydrolysis of 10% to 75%.

10. The method of claim 8, wherein the nutritional composition is a human milk fortifier.

11. The method of claim 10, wherein the nutritional composition is a powdered human milk fortifier.

12. The method claim 8, wherein the nutritional composition is an infant formula.

13. The method of claim 12, wherein the nutritional composition is a preterm infant formula.

14. A method for the prevention, delay of progression, or the treatment of a circulatory disorder characterized by inadequate blood flow to the brain, lung, or both comprising:
administering to an individual in need thereof a nutritional composition comprising a therapeutically effective amount of a DPP-IV inhibiting protein wherein the protein has a degree of hydrolysis of 10% to 75%.

15. A method of treating tissue inflammation in at least one of the gut, lung, and brain of an individual in need thereof, the method comprising administering to the individual a nutritional composition comprising a protein with a degree of hydrolysis of 10% to 75% and wherein after administration the tissue inflammation in at least one of the gut, lung, and brain is reduced.
